(19) United States
(12) Patent Application Publication
(10) Pub. No.: US 2022/0193289 A1
(43) Pub. Date: Jun. 23, 2022

(54) Autonomously Operable Sanitation Sprayer

(71) Applicant: Motivo Engineering, LLC, Gardena, CA (US)

(72) Inventors: Zachary Meyer Omohundro, Carson, CA (US); Maximilian Fredrick Ballenger Sluiter, Carson, CA (US); Xavier Kevin Dedenbach, Placentia, CA (US); Jorge Alejandro Esteves, Whittier, CA (US); Damon Christopher Pipenberg, Torrance, CA (US); Praveen V. Penmetsa, Newark, CA (US); Charles Allen Goad, Valencia, CA (US)

(21) Appl. No.: 17/556,375

(22) Filed: Dec. 20, 2021

Related U.S. Application Data
(60) Provisional application No. 63/128,396, filed on Dec. 21, 2020.

(51) Int. Cl. A61L 2/22; B05B 5/08; B05B 1/20; B05B 15/40; B05B 5/03; A61L 2/26; A61L 2/24

(52) U.S. Cl. CPC A61L 2/22; B05B 5/085; B05B 1/20; B05B 15/40; A61L 2202/14; A61L 2/26; A61L 2/24; A61L 2202/25; A61L 2202/16; B05B 5/03

(57) ABSTRACT

An apparatus and methods are provided for a remotely operable sanitation sprayer for quickly disinfecting large indoor spaces. The sanitation sprayer includes a body supported by drive wheels and one or more casters. The drive wheels are configured to rotate at different speeds to steer the sanitation sprayer. A boom comprising a generally elongate member is vertically coupled with the body. Nozzles disposed along the length of the boom are configured to disperse a disinfectant into the indoor space. The nozzles are of an electrostatic variety configured to electrostatically charge the disinfectant exiting the sanitation sprayer. The sanitation sprayer further includes electronic equipment for remotely operating the sanitation sprayer. A front of the sanitation sprayer may be equipped with one or more cameras and sensors that facilitate detecting nearby objects. The cameras and sensors may be configured provide a first-person view to a practitioner remotely operating the sanitation sprayer.

### PRIORITY

This application claims the benefit of and priority to U.S. Provisional Application No. 63/128,396, filed on Dec. 21, 2020, the entirety of which is incorporated herein by reference.

### FIELD

Embodiments of the present disclosure generally relate to electric sanitation sprayers. More specifically, embodiments of the disclosure relate to an apparatus and methods for an autonomously operable sanitation sprayer that may be remotely operated to quickly disinfect contaminated spaces.

### BACKGROUND

Microbes, such as bacteria and viruses, can be transmitted through airborne droplets and aerosols, as well as transmitted by hand-to-hand or hand-to-surface contact. Transmission of microbes, particularly highly contagious microbes, is a serious health problem and is well known to lead to infections that can spread quickly. As will be appreciated, groups of people seated or working in proximity gives rise to an increased risk of spreading dangerous contagions.

Contagious diseases such as influenza, COVID-19, and the like are affecting many procedures used by medical personnel and the general public. Infection disease specialists acknowledge that aerosols from breathing and speaking can accumulate and remain infectious in indoor air and on indoor surfaces for hours. In an attempt to combat the spread of diseases, many public venues such as retail stores, super markets, and banks are increasingly relying upon protective shields or barriers to block airborne droplets as well as using a variety of disinfection techniques to inhibit virus transmission while allowing safe access to the public.

Mass transit vehicles, such as city buses, trains, and airplanes, are particularly susceptible to entrapping and spreading airborne droplets and viral contagions. Buses, trains, and airplanes generally comprise relatively small indoor spaces, include tight seating, and may see many people entering and exiting throughout each day. As such, passengers and operators are placed at an increased risk of encountering a wide variety of dangerous contagions. Embodiments disclosed herein provide an autonomously operable sanitation sprayer and methods for quickly and remotely disinfecting potentially contaminated spaces, such as airplanes or theaters.

### SUMMARY

An apparatus and methods are provided for a remotely operable sanitation sprayer for quickly disinfecting large indoor spaces. The sanitation sprayer includes a body supported by drive wheels and one or more casters. The drive wheels are configured to rotate at different speeds to steer the sanitation sprayer. A boom comprising a generally elongate member is vertically coupled with the body. Nozzles disposed along the length of the boom are configured to disperse a disinfectant into the indoor space. The nozzles are of an electrostatic variety configured to electrostatically charge the disinfectant exiting the sanitation sprayer. The sanitation sprayer further includes electronic equipment for remotely operating the sanitation sprayer. A front of the sanitation sprayer may be equipped with one or more cameras and sensors that facilitate detecting nearby objects. The cameras and sensors may be configured provide a first-person view to a practitioner remotely operating the sanitation sprayer.

In an exemplary embodiment, a sanitation sprayer, comprises: a body supported by drive wheels and one or more casters; a boom coupled with the body; nozzles disposed along the length of the boom; a liquid tank in fluid communication with the nozzles; and an air compressor/blower system for dispersing a disinfectant stored in the liquid tank.

In another exemplary embodiment, the body has a relatively narrow width to facilitate moving along an aisle between seats, such as the seats in an airplane, a train, a bus, and the like. In another exemplary embodiment, the width of the body is similar to an airline galley cart. In another exemplary embodiment, the drive wheels are configured to rotate at different speeds so as to steer the sanitation sprayer as desired. In another exemplary embodiment, the one or more casters are of a swivel variety that allows the drive wheels to steer the sanitation sprayer in various desirable directions.

In another exemplary embodiment, the boom comprises a generally elongate member that is vertically erected with respect to the body by way of a boom support. In another exemplary embodiment, a stack light is disposed atop the boom support and configured to provide a visual indication of any of various operating modes of the sanitation sprayer. In another exemplary embodiment, the nozzles include side nozzles that are configured to spray disinfectant to the sides of the sanitation sprayer. In another exemplary embodiment, the nozzles include front nozzles that are configured to spray the disinfectant in a forward direction with respect to the sanitation sprayer. In another exemplary embodiment, the side nozzles and the front nozzles are of an electrostatic variety configured to positively or negatively charge the disinfectant exiting the sanitation sprayer.

In another exemplary embodiment, a control panel includes multiple switches and indicator lights configured to facilitate operation of the sanitation sprayer. In another exemplary embodiment, the control panel includes an E-stop pushbutton configured to enable a practitioner to immediately cease operation of the sanitation sprayer. In another exemplary embodiment, the liquid tank is configured to hold a desired volume of liquid disinfectant that is to be sprayed into an indoor space.

In another exemplary embodiment, the air compressor/blower system includes an air compressor/blower motor, a blower output plenum, and an air filter. In another exemplary embodiment, the air compressor/blower motor intakes air through the air filter and outputs the air through the blower output plenum to a manifold system that is in fluid communication with the nozzles. In another exemplary embodiment, a water pump pushes the liquid disinfectant from the liquid tank to the manifold system.

In another exemplary embodiment, the sanitation sprayer further comprises one or more modules that include electronic equipment for remotely operating the sanitation sprayer. In another exemplary embodiment, a front of the sanitation sprayer is equipped with one or more cameras and sensors that facilitate detecting nearby objects. In another exemplary embodiment, the one or more cameras provide a first-person view to a practitioner remotely operating the sanitation sprayer.

These and other features of the concepts provided herein may be better understood with reference to the drawings, description, and appended claims.

### BRIEF DESCRIPTION OF THE DRAWINGS

The drawings refer to embodiments of the present disclosure in which:

FIG. 1 illustrates an isometric view of an exemplary embodiment of an autonomously operable sanitation sprayer for disinfecting contaminated spaces according to the present disclosure;

FIG. 2 illustrates a side plan view of the autonomously operable sanitation sprayer of FIG. 1 in absence of an exterior housing comprising a body of the sanitation sprayer;

FIG. 3 illustrates an exemplary embodiment of a control panel that includes multiple switches and indicator lights configured to facilitate operation of the sanitation sprayer of FIG. 1;

FIG. 4 illustrates an exemplary-use environment comprising a practitioner wirelessly operating a sanitation sprayer by way of a remote controller, in accordance with the present disclosure;

FIG. 5 illustrates an isometric view of an exemplary embodiment of an autonomously operable sanitation sprayer for disinfecting contaminated spaces according to the present disclosure; and

FIG. 6 illustrates is a block diagram illustrating an exemplary data processing system that may be used with embodiments of an autonomously or remotely operable sanitation sprayer according to the present disclosure.

While the present disclosure is subject to various modifications and alternative forms, specific embodiments thereof have been shown by way of example in the drawings and will herein be described in detail. The present disclosure should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the autonomously operable sanitation sprayer and methods disclosed herein may be practiced without these specific details. In other instances, specific numeric references such as “first nozzle,” may be made. However, the specific numeric reference should not be interpreted as a literal sequential order but rather interpreted that the “first nozzle” is different than a “second nozzle.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

Contagious diseases such as hepatitis, influenza, and COVID-19 are affecting many procedures used by health care and medical personnel, as well as the general public. Aerosols from simply breathing and speaking can accumulate and remain infectious in indoor air and surfaces for hours. Mass transit vehicles, such as city buses, trains, and airplanes, are particularly susceptible to entrapping and spreading airborne droplets and viral contagions. Buses, trains, and airplanes generally comprise relatively small indoor spaces, include tight seating, and may see many people entering and exiting throughout each day. As such, passengers and operators are placed at an increased risk of encountering a wide variety of dangerous contagions. Embodiments disclosed herein provide a sanitation sprayer and methods for quickly and remotely disinfecting potentially contaminated spaces, such as airplanes or theaters.

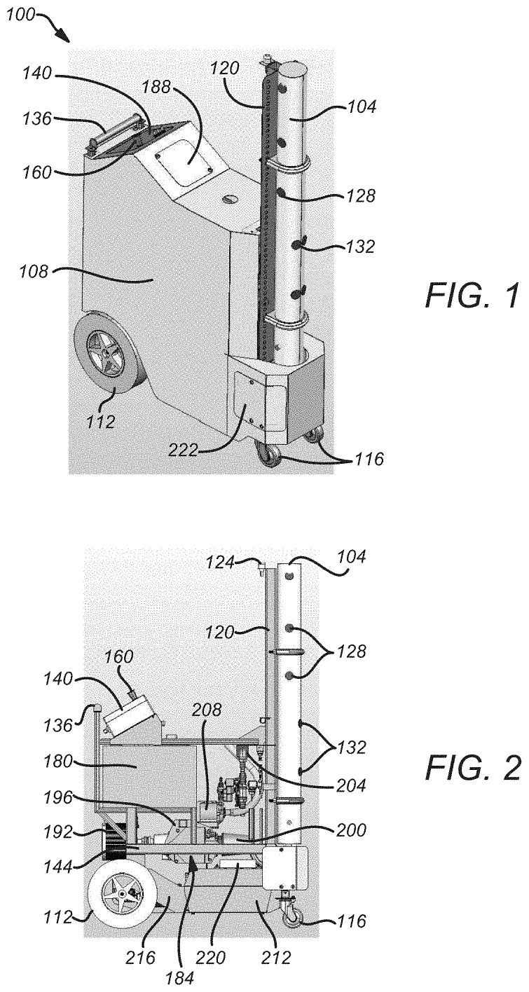

FIG. 1 illustrates an isometric view of an exemplary embodiment of an autonomously operable sanitation sprayer 100 (hereinafter, “sprayer 100”). The sprayer 100 generally is of an electrostatic variety of sanitation sprayer and is configured to quickly distribute large volumes of disinfectant into large indoor spaces. The sprayer 100 includes a boom 104 that is coupled with a body 108 supported by drive wheels 112 and one or more casters 116. The body 108 preferably has a relatively narrow width, similar to an airplane galley cart, to facilitate moving the sprayer 100 along an aisle between seats, such as the seats in an airplane, a train, a bus, and the like. The drive wheels 112 are configured to support the weight of the sprayer 100, including onboard disinfectant, as well as propel the sprayer 100. It is contemplated that the drive wheels 112 may be configured to rotate at different speeds so as to steer the sprayer 100 as desired. As will be appreciated, the casters 116 may be of a swivel variety that supports the boom 104 while allowing the drive wheels 112 to steer the sprayer 100 in various desirable directions. In some embodiments, however, the casters 116 may be configured to be electronically swiveled so as to actively steer the sprayer 100.

The boom 104 comprises a generally elongate member that is vertically erected with respect to the body 108 by way of a boom support 120. A stack light 124 may be disposed atop the boom support 120 and configured to provide a visual indication of any of various operating modes of the sprayer 100. For example, the stack light 124 may be configured to illuminate a green color to indicate a full tank of disinfectant onboard the sprayer 100. A yellow color of the stack light 124 may indicate that the tank of disinfectant is running low, and a red stack light 124 may indicate that the disinfectant tank is nearly empty. Other uses of the stack light 124 will be apparent to those skilled in the art.

The boom support 120 fixates the boom 104 in a vertical direction suitable for distributing a disinfectant spray over a large area nearby the sprayer 100. To this end, multiple side nozzles 128 are disposed along the length of the boom 104 and configured to spray the disinfectant to the sides of the sprayer 100. In the embodiment illustrated in FIG. 1, the boom 104 includes front nozzles 132 that are configured to spray the disinfectant in a forward direction with respect to the sprayer 100. In some embodiments, however, the front nozzles 132 may be omitted without limitation (see, for example, FIG. 4). It is contemplated that the side and front nozzles 128, 132 generally are of an electrostatic variety configured to positively or negatively charge the disinfectant exiting the nozzles 128, 132.

As shown in FIG. 1, the sprayer 100 includes a proximal handle 136 and a control panel 140. The proximal handle 136 generally is a rigid member that enables a practitioner to push, pull, and steer the sprayer 100 by hand, as desired. It is contemplated that the proximal handle 136 is suitable for instances wherein the sprayer 100 must be navigated by hand, such as during disinfecting a tight indoor space or in the case of a wireless system failure. In some embodiments, the proximal handle 136 comprises a portion of a chassis 144 (see FIG. 2) that supports interior components comprising the sprayer 100.

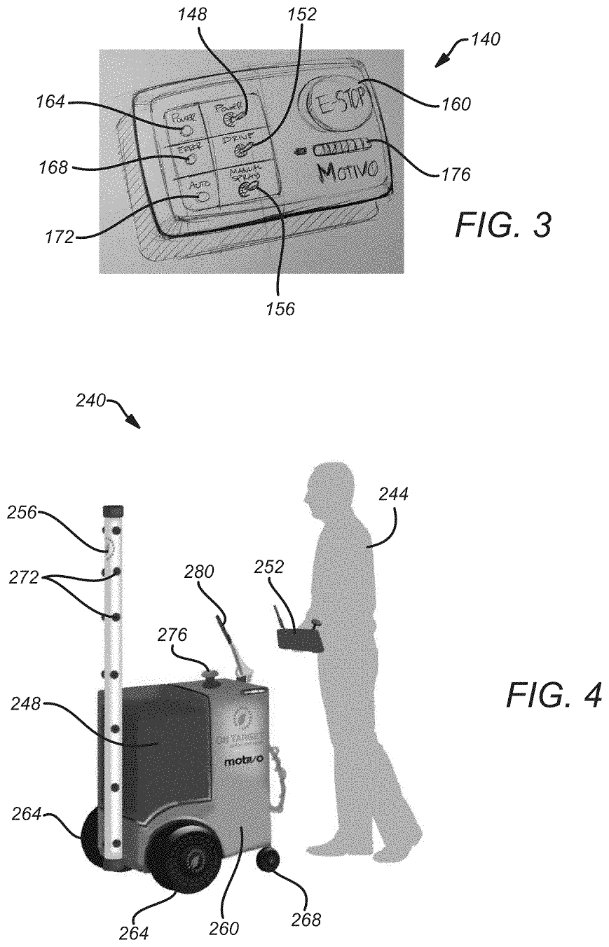

The control panel 140 includes multiple switches and indicator lights configured to facilitate operation of the sprayer 100. In an embodiment shown in FIG. 3, the control panel 140 includes a Power switch 148, a Drive switch 152, a Manual Spray switch 156, and an E-Stop pushbutton 160. As will be appreciated, the Power switch 148 enables a practitioner to turn on the sprayer 100, the Drive switch 152 enables the practitioner to engage a drive system to propel the sprayer 100, and the Manual Spray switch 156 enables the practitioner to turn off a remote spraying mode of the sprayer 100 when manual spraying is desired. The E-Stop pushbutton 160 is configured to cause the sprayer 100 to stop moving and cease spraying disinfectant. Thus, the practitioner may press the E-Stop pushbutton 160 to halt operation of the sprayer 100 in the event of a problem or error.

As further shown in FIG. 3, the control panel 140 may include a Power indicator light 164, an Error indicator light 168, an Auto indicator light 172, as well as a bar gauge 176. The power indicator light 164 may be configured to indicate with the sprayer 100 is turned and ready to begin dispersing the disinfectant. The Error indicator light 168 may be configured to illuminate in the event of any error that is detected. The Auto indicator light 172 may be configured to illuminate with a remote spraying mode of the sprayer 100 is selected. Thus, the Auto indicator light 172 may turn off when the Manual Spray switch 156 is switched on.

The bar gauge 176 may be configured to indicate a current level of any quantity that changes during operation of the sprayer 100. For example, in some embodiments, the bar gauge 176 may be configured to display a current state of charge of an onboard battery that powers the sprayer 100. In some embodiments, the bar gauge 176 may be configured to display a current amount of the disinfectant or water that is remaining within the sprayer 100. In some embodiments, the bar gauge 176 may be switchable such that the practitioner may select different quantities that are represented by the bar gauge 176. For example, in some embodiments, the bar gauge 176 may be switched among indicating the current state of charge of the onboard battery, the current amount the disinfectant, and the current amount of water remaining in the sprayer 100. Other controls, gauges, and indicators that may be incorporated into the control panel 140 will be apparent to those skilled in the art.

FIG. 2 illustrates a side plan view of the remotely operable sanitation sprayer 100 of FIG. 1 in absence of an exterior housing comprising the body 108. As shown in FIG. 2, the chassis 144 supports a liquid tank 180 and an air compressor/blower system 184. The liquid tank 180 generally is configured to hold a desired volume of liquid disinfectant that is to be sprayed into an indoor space. As shown in FIG. 1, a liquid tank lid 188 disposed in the exterior housing of the body 108 is configured to provide access to the liquid tank 180. As will be appreciated, the liquid tank lid 188 facilitates filling the liquid tank 180 with the liquid disinfectant that is to be sprayed into the indoor space.

The air compressor/blower system 184 generally is configured to provide air pressure to atomize liquid disinfectant being dispersed by the nozzles 128, 132. In the illustrated embodiment of FIG. 2, the air compressor/blower system 184 includes an air compressor/blower motor 192, a blower output plenum 196 and an air filter 200. Those skilled in the art will recognized that the air compressor/blower motor 192 generally intakes air through the air filter 200 and outputs the air through the blower output plenum 196 to a manifold system 204 and the nozzles 128, 132. In the illustrated embodiment, a water pump 208 pushes the liquid disinfectant from the liquid tank 180 to the manifold system 204 and the nozzles 128, 132.

With continuing reference to FIG. 2, a battery box 212 and one or more traction motors 216 are disposed at a bottom of the sprayer 100. In general, the battery box 212 encloses one or more rechargeable onboard batteries for powering the sprayer 100. As shown in FIG. 1, a charger port cover 222 may be used to access a charging port whereby the onboard batteries may be recharged. The traction motors 216 are in mechanical communication with the drive wheels 112 for propelling and steering the sprayer 100. In the embodiment illustrated in FIG. 2, each drive wheel 112 is driven by a dedicated traction motor 216. For example, a right-hand traction motor 216 may power the drive wheel 112 on the right-hand side of the sprayer 100, and a left-hand traction motor 216 may power the drive wheel 112 on the left-hand side of the sprayer 100. As such, the right- and left-hand traction motors 216 may be powered independently so as to drive wheels 112 at different rotational speeds for the purpose of steering the sprayer 100. Those skilled in the art will recognize that the dedicated traction motors 216 eliminate any need for incorporating differential gears into the sprayer 100.

In the illustrated embodiment of FIG. 2, one or more modules 220 are mounted atop the battery box 212. It is contemplated that the modules 220 include circuitry that is configured to operate the various components comprising the sprayer 100. For example, in some embodiments, the modules 220 may include controllers for the air compressor/blower motor 192, the water pump 208, and the traction motors 216.

In some embodiments, the modules 220 may include electronic equipment for remotely operating the sprayer 100. For example, FIG. 4 illustrates an exemplary-use environment 240 wherein a practitioner 244 wirelessly operates a remotely operable sanitation sprayer 248 by way of a remote controller 252. The sprayer 248 shown in FIG. 4 is similar to the sprayer 100 shown in FIG. 1. As such, the sprayer 248 includes a boom 256 that is coupled with a body 260 supported by drive wheels 264 at a front of the sprayer 248 and a pair of casters 268 at a rear of the sprayer 248. The drive wheels 264 are configured to support the weight of the sprayer 248, including onboard disinfectant, as well as to propel and steer the sprayer 248. As described hereinabove, the drive wheels 264 may be configured to rotate at different speeds so as to steer the sprayer 248 as desired. The casters 268 may be of a swivel variety that allows the drive wheels 264 to steer the sprayer 248 into tight turns.

The boom 256 generally is an elongate member that is vertically erected with respect to the body 260 and is configured for distributing a disinfectant spray over a large area nearby the sprayer 248. Multiple nozzles 272 are disposed along the length of the boom 256 and configured to disperse the disinfectant to the sides of the sprayer 248. In some embodiments, the boom 256 may further include front nozzles, such as the front nozzles 132, that are configured to spray a portion of the disinfectant in a forward direction with respect to the sprayer 248. It is contemplated that the nozzles 272 generally are of an electrostatic variety configured to positively or negatively charge the disinfectant exiting the nozzles 128, 132. Further, the sprayer 248 preferably includes an E-Stop pushbutton 276 that enables a practitioner to immediately cease operation of the sprayer 248 in the event of a problem or error.

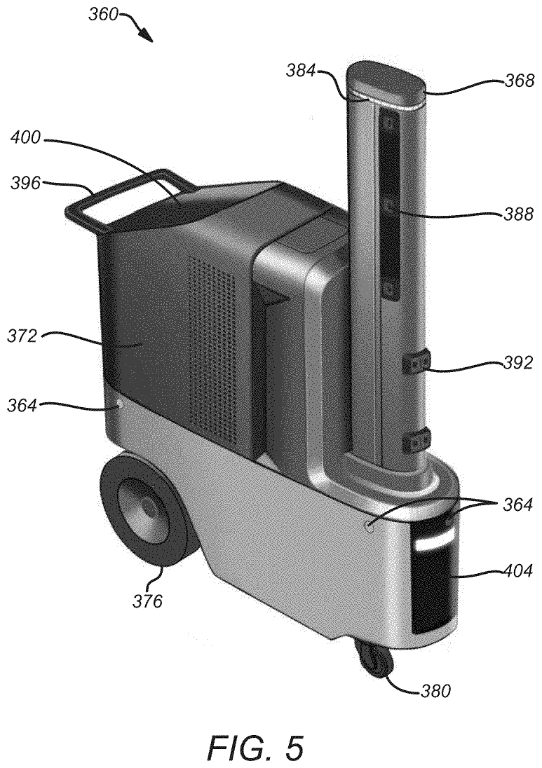

As further shown in FIG. 4, the sprayer 248 includes an antenna 280 that is configured to enable wireless communication with the remote controller 252. Further, the sprayer 248 may include one or more devices configured to give the sprayer 248 remote detection capabilities. For example, a front of the sprayer 248 may be equipped with cameras and sensors that facilitate detecting nearby objects. In some embodiments, the cameras may provide a first-person view (FPV) to the practitioner 244 operating the sprayer 248, or the cameras may enable an onboard artificial intelligence to detect targeted objects, conditions, and obstructions nearby a desired path. It is contemplated, that in some embodiments, the sensors may be configured to enable the sprayer 248 to utilize electromagnetic wavelengths outside the visible light spectrum, such as Infrared wavelengths and ultrasonic sensors to facilitate operation of the sprayer 248. For example, FIG. 5 illustrates an exemplary embodiment of an autonomous sanitation sprayer 360 that includes ultrasonic sensors 364 that may be configured to provide obstruction and distance detection so as to assist with remote operation of the sprayer 360. In some embodiments, the ultrasonic sensors 364 may be configured to assist with detecting potential obstacles and avoiding collisions, as well as centering the sprayer 360 in an aisle between seats, such as the seats in an airplane, a train, a bus, and the like. Further, in some embodiments, the ultrasonic sensors 364 may be configured to operate as secondary sensors while the sprayer 360 is operating autonomously, as described herein.

As will be appreciated, the sprayer 360 shown in FIG. 5 is substantially similar to the sprayer 100 of FIG. 1. The sprayer 360 generally is of an electrostatic variety of sanitation sprayer that includes a boom 368 coupled with a body 372 that is supported by drive wheels 376 and one or more casters 380. The body 372 has an advantageously narrow width, similar to an airplane galley cart, to facilitate maneuvering the sprayer 360 along an aisle between seats, such as the seats in an airplane, a train, a bus, and the like. The drive wheels 376 are configured to support the weight of the sprayer 360, including onboard disinfectant, as well as propel the sprayer 360. The drive wheels 376 may be configured to rotate at different speeds so as to steer the sprayer 360 as desired. Further, the casters 380 may be of a swivel variety that supports the boom 368 while allowing the drive wheels 376 to maneuver the sprayer 360 in various desirable directions. In some embodiments, the casters 380 may be configured to be electronically swiveled so as to actively steer the sprayer 360.

The boom 368 is a generally elongate member that is vertically erected with respect to the body 372. A stack light 384 may be incorporated into a top of the boom 368 and configured to provide a visual indication of any of various operating modes of the sprayer 360, as described herein with respect to FIG. 1. As will be appreciated, the vertical orientation of the boom 368 is advantageous for distributing a disinfectant spray over a large area nearby the sprayer 360. Multiple side nozzles 388 may be disposed along the length of the boom 368 and configured to spray the disinfectant to the sides of the sprayer 360. Further, the boom 368 may include front nozzles 392 that are configured to spray the disinfectant in a forward direction with respect to the sprayer 360. As mentioned hereinabove, the side and front nozzles 388, 392 generally are of an electrostatic variety configured to positively or negatively charge the disinfectant exiting the nozzles 388, 392.

As shown in FIG. 5, the sprayer 360 includes a proximal handle 396 and a control panel 400. The proximal handle 396 generally enables a practitioner to desirably maneuver the sprayer 360 by hand, such as during disinfecting a tight indoor space or in the case of a wireless system failure. The control panel 400 generally includes multiple switches and indicator lights configured to facilitate operation of the sprayer 360. As described herein with respect to FIG. 3, the control panel 400 may include any one or more of a Power switch, a Drive switch, a Manual Spray switch, and an Emergency-Stop pushbutton. As will be appreciated, the Power switch enables a practitioner to turn on the sprayer 360, the Drive switch enables the practitioner to engage a drive system to propel the sprayer 360, and the Manual Spray switch enables the practitioner to turn off a remote spraying mode of the sprayer 360 when manual spraying is desired. The Emergency-Stop pushbutton may be configured to cause the sprayer 360 to stop moving and cease spraying disinfectant.

As mentioned herein, the sprayer 360 may be equipped with ultrasonic sensors 364 and medium-range sensors 404 that facilitate detecting nearby objects. It is contemplated that the ultrasonic sensors 364 may be configured to provide obstruction and distance detection so as to assist with remote operation of the sprayer 360. In some embodiments, the ultrasonic sensors 364 may be configured to assist with detecting potential obstacles and avoiding collisions, as well as centering the sprayer 360 in an aisle between seats, such as the seats in an airplane, a train, a bus, and the like. Further, in some embodiments, the ultrasonic sensors 364 may be configured to operate as secondary sensors while the sprayer 360 is operating autonomously.

The medium-range sensors 404 generally are configured to support an autonomous functionality of the sprayer 360. It is contemplated that an onboard artificial intelligence may be configured to be used the medium-range sensors 404 to identify objects in front of the sprayer 360 so as to avoid running into the objects. The medium-range sensors 404 may comprise any of stereoscopic cameras, monocular cameras, ultrasonic sensors, Infrared sensors, Lidar sensors, and the like. In some embodiments, the onboard artificial intelligence may be configured to use the medium-range sensors 404 to override commands of a remote practitioner in the event that the onboard artificial intelligence detects an object or a person obstructing the trajectory of the sprayer 360.

Moreover, it is contemplated that the onboard artificial intelligence may be configured to collect date while the sprayer 360 is operating and push the data to a client software application or the cloud by way of an onboard wireless connection, such as a Wi-Fi or 4G connection. As will be appreciated, the data collected during disinfecting an area may serve to ensure traceability of the sprayer 360. For example, the collected data may be used to demonstrate to a client that the sprayer 360 sanitized a specific public space at given time by using a certain amount of disinfectant. Further, the location of the specific public space may be confirmed by way of the geolocation of the sprayer 360.

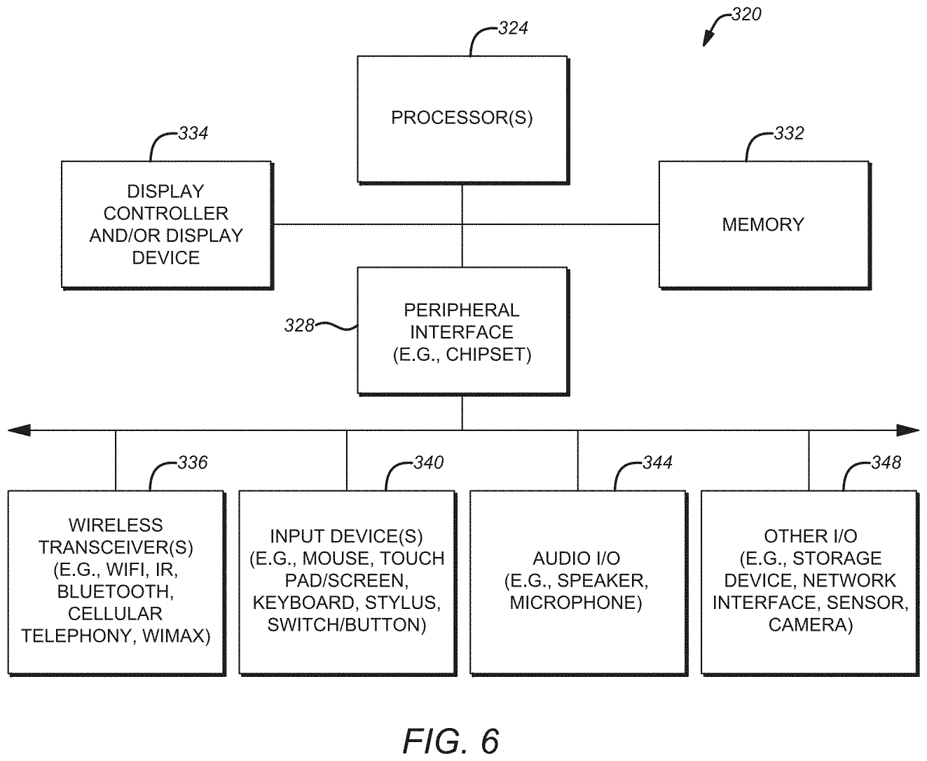

As will be appreciated, the sprayer 360 generally houses circuitry, including one or more processors, configured to run software applications suitable for operating the sprayer 360, including the above-mentioned sensors 364, 404. To this end, FIG. 6 is a block diagram illustrating an exemplary data processing system 320 that may be used in conjunction with the sprayer 360 to perform any of the processes or methods described herein. System 320 may represent circuitry within the body 372 of the sprayer 360, a desktop, a tablet, a server, a mobile phone, a personal digital assistant (PDA), a personal communicator, a network router or hub, a wireless access point (AP) or repeater, a set-top box, or any combination thereof.

In an embodiment, illustrated in FIG. 6, system 320 includes a processor 324 and a peripheral interface 328, also referred to herein as a chipset, to couple various components to the processor 324, including a memory 332 and devices 336-348 via a bus or an interconnect. Processor 324 may represent a single processor or multiple processors with a single processor core or multiple processor cores included therein. Processor 324 may represent one or more general-purpose processors such as a microprocessor, a central processing unit (CPU), or the like. More particularly, processor 324 may be a complex instruction set computing (CISC) microprocessor, reduced instruction set computing (RISC) microprocessor, very long instruction word (VLIW) microprocessor, or processor implementing other instruction sets, or processors implementing a combination of instruction sets. Processor 324 may also be one or more special-purpose processors such as an application specific integrated circuit (ASIC), a field programmable gate array (FPGA), a digital signal processor (DSP), a network processor, a graphics processor, a network processor, a communications processor, a cryptographic processor, a co-processor, an embedded processor, or any other type of logic capable of processing instructions. Processor 324 is configured to execute instructions for performing the operations and steps discussed herein.

Peripheral interface 328 may include a memory control hub (MCH) and an input output control hub (ICH). Peripheral interface 328 may include a memory controller (not shown) that communicates with a memory 332. The peripheral interface 328 may also include a graphics interface that communicates with graphics subsystem 334, which may include a display controller and/or a display device. The peripheral interface 328 may communicate with the graphics device 334 by way of an accelerated graphics port (AGP), a peripheral component interconnect (PCI) express bus, or any other type of interconnects.

An MCH is sometimes referred to as a Northbridge, and an ICH is sometimes referred to as a Southbridge. As used herein, the terms MCH, ICH, Northbridge and Southbridge are intended to be interpreted broadly to cover various chips that perform functions including passing interrupt signals toward a processor. In some embodiments, the MCH may be integrated with the processor 324. In such a configuration, the peripheral interface 328 operates as an interface chip performing some functions of the MCH and ICH. Furthermore, a graphics accelerator may be integrated within the MCH or the processor 324.

Memory 332 may include one or more volatile storage (or memory) devices, such as random access memory (RAM), dynamic RAM (DRAM), synchronous DRAM (SDRAM), static RAM (SRAM), or other types of storage devices. Memory 332 may store information including sequences of instructions that are executed by the processor 324, or any other device. For example, executable code and/or data of a variety of operating systems, device drivers, firmware (e.g., input output basic system or BIOS), and/or applications can be loaded in memory 332 and executed by the processor 324. An operating system can be any kind of operating systems, such as, for example, Windows® operating system from Microsoft®, Mac OS®/iOS® from Apple, Android® from Google®, Linux®, Unix®, or other real-time or embedded operating systems such as VxWorks.

Peripheral interface 328 may provide an interface to IO devices, such as the devices 336-348, including wireless transceiver(s) 336, input device(s) 340, audio IO device(s) 344, and other IO devices 348. Wireless transceiver 336 may be a WiFi transceiver, an infrared transceiver, a Bluetooth transceiver, a WiMax transceiver, a wireless cellular telephony transceiver, a satellite transceiver (e.g., a global positioning system (GPS) transceiver) or a combination thereof. Input device(s) 340 may include a mouse, a touch pad, a touch sensitive screen (which may be integrated with display device 334), a pointer device such as a stylus, and/or a keyboard (e.g., physical keyboard or a virtual keyboard displayed as part of a touch sensitive screen). For example, the input device 340 may include a touch screen controller coupled with a touch screen. The touch screen and touch screen controller can, for example, detect contact and movement or break thereof using any of a plurality of touch sensitivity technologies, including but not limited to capacitive, resistive, infrared, and surface acoustic wave technologies, as well as other proximity sensor arrays or other elements for determining one or more points of contact with the touch screen.

Audio IO 344 may include a speaker and/or a microphone to facilitate voice-enabled functions, such as voice recognition, voice replication, digital recording, and/or telephony functions. Other optional devices 348 may include a storage device (e.g., a hard drive, a flash memory device), universal serial bus (USB) port(s), parallel port(s), serial port(s), a printer, a network interface, a bus bridge (e.g., a PCI-PCI bridge), sensor(s) (e.g., a motion sensor, a light sensor, a proximity sensor, etc.), or a combination thereof. Optional devices 348 may further include an imaging processing subsystem (e.g., a camera), which may include an optical sensor, such as a charged coupled device (CCD) or a complementary metal-oxide semiconductor (CMOS) optical sensor, utilized to facilitate camera functions, such as recording photographs and video clips.

Note that while FIG. 6 illustrates various components of a data processing system, it is not intended to represent any particular architecture or manner of interconnecting the components, as such details are not germane to embodiments of the present disclosure. It should also be appreciated that network computers, handheld computers, mobile phones, and other data processing systems, which have fewer components or perhaps more components, may also be used with embodiments of the invention disclosed hereinabove.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it should be appreciated that throughout the description, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system's memories or registers or other such information storage, transmission or display devices.

The techniques shown in the figures can be implemented using code and data stored and executed on one or more electronic devices. Such electronic devices store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals—such as carrier waves, infrared signals, digital signals).

The processes or methods depicted in the preceding figures may be performed by processing logic that comprises hardware (e.g. circuitry, dedicated logic, etc.), firmware, software (e.g., embodied on a non-transitory computer readable medium), or a combination of both. Although the processes or methods are described above in terms of some sequential operations, it should be appreciated that some of the operations described may be performed in a different order. Moreover, some operations may be performed in parallel rather than sequentially.

While the autonomously operable sanitation sprayer and methods have been described in terms of particular variations and illustrative figures, those of ordinary skill in the art will recognize that the autonomously operable sanitation sprayer is not limited to the variations or figures described. In addition, where methods and steps described above indicate certain events occurring in certain order, those of ordinary skill in the art will recognize that the ordering of certain steps may be modified and that such modifications are in accordance with the variations of the autonomously operable sanitation sprayer. Additionally, certain of the steps may be performed concurrently in a parallel process when possible, as well as performed sequentially as described above. To the extent there are variations of the autonomously operable sanitation sprayer, which are within the spirit of the disclosure or equivalent to the autonomously operable sanitation sprayer found in the claims, it is the intent that this patent will cover those variations as well. Therefore, the present disclosure is to be understood as not limited by the specific embodiments described herein, but only by scope of the appended claims.

What is claimed is:

1. A sanitation sprayer, comprising:
a body supported by drive wheels and one or more casters;
a boom coupled with the body;
nozzles disposed along the length of the boom;
a liquid tank in fluid communication with the nozzles; and
an air compressor/blower system for dispersing a disinfectant stored in the liquid tank.

2. The sanitation sprayer of claim 1, wherein the body has a relatively narrow width to facilitate moving along an aisle between seats, such as the seats in an airplane, a train, a bus, and the like.

3. The sanitation sprayer of claim 2, wherein the width of the body is similar to an airline galley cart.

4. The sanitation sprayer of claim 1, wherein the drive wheels are configured to rotate at different speeds so as to steer the sanitation sprayer as desired.

5. The sanitation sprayer of claim 4, wherein the one or more casters are of a swivel variety that allows the drive wheels to steer the sanitation sprayer in various desirable directions.

6. The sanitation sprayer of claim 1, wherein the boom comprises a generally elongate member that is vertically erected with respect to the body by way of a boom support.

7. The sanitation sprayer of claim 6, wherein a stack light is disposed atop the boom support and configured to provide a visual indication of any of various operating modes of the sanitation sprayer.

8. The sanitation sprayer of claim 1, wherein the nozzles include side nozzles that are configured to spray disinfectant to the sides of the sanitation sprayer.

9. The sanitation sprayer of claim 8, wherein the nozzles include front nozzles that are configured to spray the disinfectant in a forward direction with respect to the sanitation sprayer.

10. The sanitation sprayer of claim 9, wherein the side nozzles and the front nozzles are of an electrostatic variety configured to positively or negatively charge the disinfectant exiting the sanitation sprayer.

11. The sanitation sprayer of claim 1, wherein a control panel includes multiple switches and indicator lights configured to facilitate operation of the sanitation sprayer.

12. The sanitation sprayer of claim 11, wherein the control panel includes an E-stop pushbutton configured to enable a practitioner to immediately cease operation of the sanitation sprayer.

13. The sanitation sprayer of claim 1, wherein the liquid tank is configured to hold a desired volume of liquid disinfectant that is to be sprayed into an indoor space.

14. The sanitation sprayer of claim 1, wherein the air compressor/blower system includes an air compressor/blower motor, a blower output plenum, and an air filter.

15. The sanitation sprayer of claim 14, wherein the air compressor/blower motor intakes air through the air filter and outputs the air through the blower output plenum to a manifold system that is in fluid communication with the nozzles.

16. The sanitation sprayer of claim 15, wherein a water pump pushes the liquid disinfectant from the liquid tank to the manifold system.

17. The sanitation sprayer of claim 1, further comprising one or more modules that include electronic equipment for remotely operating the sanitation sprayer.

18. The sanitation sprayer of claim 1, wherein a front of the sanitation sprayer is equipped with one or more cameras and sensors that facilitate detecting nearby objects.

19. The sanitation sprayer of claim 18, wherein the one or more cameras provide a first-person view to a practitioner remotely operating the sanitation sprayer.
